(19) United States
(12) Patent Application Publication
(10) Pub. No.: US 2017/0052749 A1
(43) Pub. Date: Feb. 23, 2017

(54) SMART DISPLAY WEAR

(71) Applicant: Seung Woo LEE, Seoul (KR)

(72) Inventor: Seung Woo LEE, Seoul (KR)

(21) Appl. No.: 15/118,531

(22) PCT Filed: Apr. 11, 2014
(86) PCT No.: PCT/KR2014/003128

§ 371 Date: Aug. 12, 2016

(30) Foreign Application Priority Data
Feb. 12, 2014 (KR) 10-2014-0016110
Feb. 13, 2014 (KR) 10-2014-0016783

(51) Int. Cl. G06F 3/14; A41D 3/00; G09G 5/36; A61B 5/00; G06F 1/16; G06F 3/041; A41D 1/00; A61B 5/01

(52) U.S. Cl. CPC G06F 3/1423; A41D 1/002; A41D 3/00; A61B 5/01; A61B 5/742; A61B 5/6804; G06F 1/1652; G06F 3/041; G09G 5/36; G06F 3/16

(57) ABSTRACT

The present invention relates to smart wear using a flexible display panel and, particularly, the present invention comprises: clothes provided through any one method between a method by which the flexible display panel is attached and fixed to a portion of or over the entire outside of a lining of the clothes or a method by which the flexible display panel forms the clothes; and a control unit for storing design information containing various colors or textures for each pan of the clothes as a video or images and outputting the design information to the flexible display panel according to settings. Accordingly, various designs can be implemented for clothes having the same shape, and thus a higher degree of design freedom can be provided to a designer so as to increase the value of the clothes, and a person who wears the clothes can create various styles with the same clothes.

### TECHNICAL FIELD

The present invention relates to smart wear, and in particular, to smart display wear in which a flexible display panel is provided on an outside thereof to enable various design implementations, add value, detect and display the user's state, and connect with various external devices, thereby maximizing the user's safely and convenience.

### DISCUSSION OF RELATED ART

The growth of display technology leads to development of flexible displays or transparent displays. Among others, flexible displays refer to bendable displays.

Flexible displays have a plastic film instead of glass substrate surrounding the liquid crystal layer used to be used in conventional liquid crystal displays (LCDs) or organic light emitting displays (OLEDs) to be flexibly bent or unbent.

Further, flexible displays are thin, lightweight, high impact-resistance, and bendable, so that they have various applications that cannot be achieved by existing glass-based displays.

For example, it is attempted to apply flexible displays to brand-new portable IT products, such as smart cards enabling real-time information check, mini personal computers (PCs) with a display bendable or reliable to be carried, as well as e-book industry that may replace magazines. textbooks, comic books or other paper-based publications.

Meanwhile, smart wear, a product of the ubiquitous era, are next-generation wear having various daily-necessity digital devices integrated with clothes and are fabric-digital convergence clothing products.

Currently increased demand for functional wear brings to the market various types of clothes for specialized use beyond existing purposes, such as protecting body or decoration.

Examples include lightweight, durable, and high-porous sportswear, fire retardant wear or pilot wear having flame retardant capability, cold weather clothing having an air layer to enforce heat retaining capability, or battle uniform for military purposes.

However, such application of flexible display panels to clothing is limited for specialized purposes, such as what is disclosed in Korean Patent Application Publication No. 2011-0011266 titled “surrounding color-sensitive camouflage.

### SUMMARY

Accordingly, an object of the present invention is to provide smart display wear that allows for various design implementations on clothes with the same shape, so as to present an enhanced degree of freedom to the designer and allows the user to enjoy the same clothing in a diversity of stylish manners.

Further, according to another embodiment of the present invention, there are provided smart display wear that add a temperature sensor or location sensor to enable the user to check his health condition or location thereby leading to increased safely.

Further, there are provided smart display wear that may wiredly or wirelessly connect with various external devices via a connector, thereby maximizing user convenience.

The above object is achieved by smart display wear comprising a garment having a flexible display panel attached or fastened onto a whole or part of an outside of a lining of the garment or configuring the garment itself; and a controller storing, as an image or video, design information including various colors or textures for each portion of the garment and outputting the design information to the flexible display panel according to a setting.

Here, the flexible display panel may be divided into a plurality of panel areas independently separated from each other, and the controller may independently control each of the panel areas.

The smart display wear may further comprise a temperature sensor provided at, at least, one or more positions on the lining or the flexible display panel to measure a body temperature of a wearer of the garment, wherein the controller displays a temperature measurement sensed by the temperature sensor at a predetermined position on the flexible display panel.

When the temperature measurement is not less than a set temperature previously entered and stored, the controller may display a color, flickering, or other alerts on the flexible display panel to externally notify.

The smart display wear may comprise a connector connectible with an external device by one or more wired or wireless methods, a touchscreen provided on a side of the garment to conduct input and output on the external device, a microphone for inputting a voice, and a speaker for outputting a voice.

The smart display wear may further comprise an output unit for externally outputting an image and voice outputted from the external device by the touchscreen.

The smart display wear may further comprise a connector connectible with an external device by one or more wired or wireless methods and motion sensors provided at multiple positions of the garment to detect a wearer's motion, wherein the wearer's motion detected by the motion sensors may be delivered to an external device via the connector.

The smart display wear may further comprise a location sensor provided on a side of the garment to detect a location of the garment; and a transceiver externally transmitting a result of detection by the location sensor so that a location of the wearer may be identified.

The smart display wear may further comprise a caller allowing a wearer to select an emergency call by a method including one or more of a voice or button manipulation, wherein when the caller is selected, the controller may externally transmit the result of detection by the location sensor, along with a call signal, via the transceiver.

The smart display wear may further comprise a power supply supplying power to the flexible display panel, wherein the power supply may be used by any one of methods for detachably exchanging, charging via a wired connection with an external power source, charging by a solar cell or charging by a tiny wind turbine.

The smart display wear may further comprise a connector connectible with an external device by one or more wired or wireless methods to enable transmission or reception of the design information with the connected external device.

The smart display wear may further include a first signaling unit provided on a side of the garment and a second signaling unit detachably provided in a belonging, such as a wallet, put in the garment and connected with the first signaling unit only within a predetermined distance.

When the first signaling unit loses connection with the second signaling unit, the controller may determine that the belonging is lost and display the same on the flexible display panel.

Accordingly, according to the present invention, the smart display wear allows for various design implementations on clothes with the same shape, so as to present an enhanced degree of freedom to the designer and allows the user to enjoy the same clothing in a diversity of stylish manners.

Further, according to another embodiment of the present invention, the smart display wear may add a temperature sensor or location sensor to enable the user to check his health condition or location thereby leading to increased safety.

Further, the smart display wear may wiredly or wirelessly connect with various external devices via a connector, thereby maximizing user convenience.

### BRIEF DESCRIPTION OF DRAWINGS

FIG. 1 is a block diagram illustrating a configuration of smart display wear according to an embodiment of the present invention;

FIG. 2 is a perspective view illustrating smart display wear according to an embodiment of the present invention.

### DESCRIPTION OF EXEMPLARY EMBODIMENTS

Hereinafter, the present invention is described in detail with reference to the accompanying drawings.

General terms widely known in the art considering the functionality of the present invention are described as the terms used herein, but the terms are not limited to those used herein, and various changes may be made thereto depending on the intention of one of ordinary skilled in the art or case laws or advent of new technology. Further, terms arbitrarily selected by the applicant of this application, if any, are described in detail in the relevant parts in the detailed description. Accordingly, the terms used herein should be determined based on their meanings and the overall disclosure, rather than by the terms themselves.

When an element “includes” another element, the element may further include the other element, rather excluding the other element, unless particularly stated otherwise. Further, the terms “unit,” “module,” or “part” as used herein denote a unit processing at least one function or operation, and a unit, module, or part may be implemented in hardware, software, or a combination thereof

The present invention described below in specific embodiments thereof is not limited to the embodiments, and various changes may be made thereto.

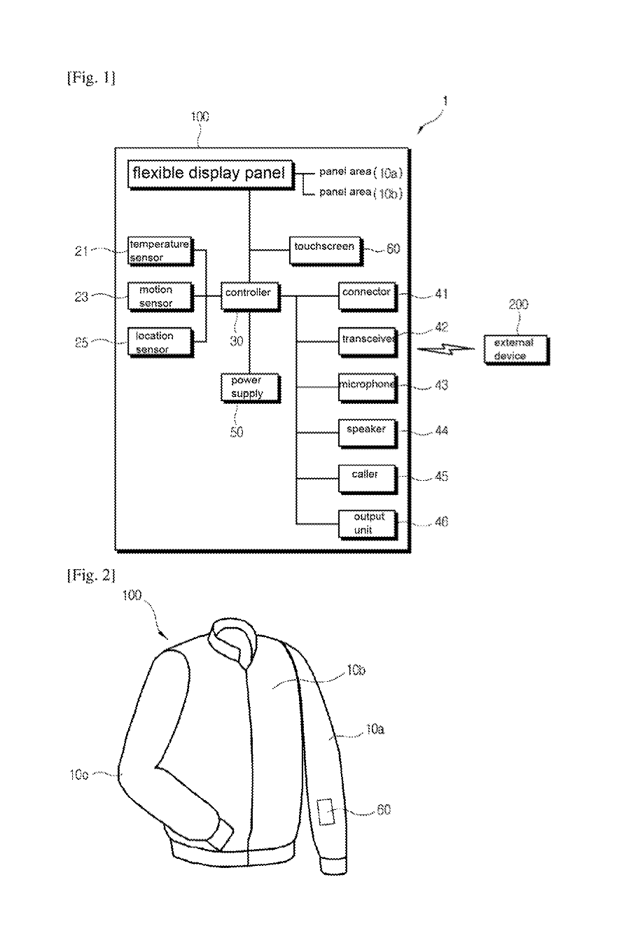

Referring to FIGS. 1 and 2, according to an embodiment of the present invention, smart display wear 1 includes a garment 100 including a flexible display panel 10 and a controller 30 outputting set design information to the flexible display panel 10.

The garment 100 is prepared using the flexible display panel 10. For example, the garment 100 may be prepared by attaching the flexible display panel 10 onto the whole or part of an outside of a lining or by cutting the flexible display panel 10 itself.

The flexible display panel 10 collectively refers to bendable or foldable display panels and may use various known types of flexible display panels. Meanwhile, the flexible display panel 10 may be used exposed to the outside of the garment 100, and thus, the flexible display panel 10 may undergo an additional process, such as waterproof coating or adding a waterproof member.

Meanwhile, as shown in FIG. 2, the flexible display panel 10 may be divided into a plurality of panel areas 10a, 10b, and 10c independently separated from each other, and in this case, the controller 30 may independently control each panel area 10a, 10b, and 10c.

In other words, in a case where the garment 100 is a normal garment, a front part 10b and a sleeve 10a may be provided to have different materials or colors, and when the controller 30 represents a color, the controller 30 may independently control the panel areas 10a and 10c and the panel area 10b, respectively, to output different colors.

Here, the panel areas 10a, 10b, and 10c are ones for diversifying and making easier the output by the controller 30, and since there is no such limitation that one panel area 10a, I Ob, and 10c should represent the same color or texture, different colors, textures, letters, images, or videos may be outputted from one panel area (e.g., panel area 10b).

Meanwhile, although the flexible display panel 10 is provided on the overall outside of the garment 100 as described above, the flexible display panel 10 may be provided only on a side of the lining as well. For example, in a case where the garment 100 is provided as a jacket as shown in FIG. 2, the flexible display panel 10 may be provided only on a part thereof which needs to be emphasized, such as a side of the front part, back number on the rear surface, or a logo.

In each case mentioned above, an electromagnetic wave blocking member may be provided inside the garment 100 in order to block harmful electromagnetic waves that may be generated from the flexible display panel 10. The electromagnetic wave blocking member may be prepared of, e.g., electromagnetic wave blocking fiber obtained by, e.g., plating a normal fiber with nickel or copper to protect the user's body from electromagnetic waves.

The controller 30 outputs design information to the flexible display panel 10.

Here, the design information may include information on the outer appearance of the garment 100, including, e.g., various colors or textures on each area of the garment 100, and the design information may be provided as an image (still image) or video (dynamic or motion image or picture).

For example, upon designing a garment, various designs depending on the shape or color of an inside of the garment in the same shape as well as a shape design for an outer part of the garment may be possible.

In this case, the design information includes various contents regarding the shape or color in the same shape rather than the shape of the garment 100. Specifically, the design information may include information on a line distinction or color distinction in a shape or information, such as color or letter(s) in each distinction, which may be a still image or an image transformed over time.

Accordingly, according to the present invention, the smart display wear allows for various design implementations on clothes with the same shape, so as to present an enhanced degree of freedom to the designer and allows the user to enjoy the same clothing in a diversity of stylish manners.

New design information may be added to the design information via connection with the external device 200. or the design information may be sent to the external device 200. Further, the respective wearers of different garments 100 may exchange or share design information.

Hereinafter, embodiments of the present invention are described in detail.

According to the present invention, the smart display wear 1 may include various sensors to enhance the user's safety and convenience.

For example, according to an embodiment of the present invention, the smart display wear 1 may include a temperature sensor 21. Such temperature sensors 21 may be placed at, at least, one or more positions in the lining or the flexible display panel 10 to measure the wearer's body temperature.

For example, the temperature sensor 21 may be placed on any one of the wearer's neck, abdomen, or back where it may directly contact the wearer's skin or may be provided at a plurality of positions to correct the temperature measurement at each position to take a temperature close to the wearer's actual temperature.

Temperature sensing by the temperature sensor 21 may be performed in various manners. For example, temperature sensing may be done at every set period or continuously.

A result sensed by the temperature sensor 21 may be displayed in various manners by the controller 30. For example, the controller 30 may display a temperature measurement sensed by the temperature sensor 21 at a predetermined position on the flexible display panel 10.

Or, the controller 30 may receive a set temperature, and when the temperature measurement is not more than the set temperature, the controller 30 may abstain from displaying it on the flexible display panel 10. and only when the temperature measurement is not less than the set temperature, the controller 30 may display it on the flexible display panel 10 by a color, or flickering or other alerts to externally notify along with the temperature measurement.

Accordingly, in an exemplary case where the smart display wear 1 according to the present invention is adopted as patient gowns, although nurses or patients themselves do not read the patients' body temperature, they may be aware of the patients' condition or state by simply checking the temperature or color shown on the patient gowns.

Further, when the body temperature measured shows an abnormality, it may be sent to a hospital system via a transceiver 42 described below, allowing for effective treatment for an emergency.

Here, the temperature sensor 21 may be provided for the purpose of measuring outside temperature as well as the wearer's temperature. In this case, a temperature measurement may be displayed on a side of the flexible display panel 10 or a color change according to the outside temperature measured may be displayed on a predetermined panel area 10a, 10b, or 10c.

Further, a sensor for measuring blood pressure may be attached on a sleeve or forearm part of the garment 100, or a sensor for detecting the amount of blood flow or heart rate may be added to continuously check the wearer's condition or state.

According to another embodiment, according to the present invention the smart display wear 1 may include a connector 41 that may access the external device 200 by one or more wired or wireless methods.

The external device 200 may be a personal terminal accessible wiredly (e.g., via a USB cable) or wirelessly (e.g., via Bluetooth), such as a smartphone, tablet PC, computer, or game player, or the external device 200 may be a mobile communication server or a service server installed in a fire station or hospital.

For example, in the case where the external device 200 is a smartphone, the smartphone may be automatically connected with the connector 41 via Bluetooth by simply putting the smartphone in a pocket of the garment 100 (e.g., by placing the smartphone within a predetermined distance from the garment 100), or the smartphone may be connected by plugging in a USB cable provided in a pocket of the garment 100.

In the latter case, a power supply 50 provided in the garment 100 may charge the smartphone via the USB cable.

A touchscreen 60 is provided on a side of the garment 100 lo enable input or output on the external device 200.

In other words, if connected with the external device 200 by way of the connector 41, the controller 30 may enable input and output on the external device 200 to be performed by the touchscreen 60.

Here, although it is described that the flexible display panel 10 performs output alone and the touchscreen 60 performs input and output both, the present invention is not limited thereto. In a case where the flexible display panel 10 itself is provided to perform both input and output, the touchscreen 60 in the instant embodiment may be regarded as one panel area 10a, 10b, or 10c.

The touchscreen 60 may be provided at a position where the wearer may easily conduct input and output. For example, as shown in FIG. 2. in a case where the garment 100 is a jacket, the touchscreen 60 may be provided on any one of the left or right sleeve.

Accordingly, the wearer may do text search or Internet browsing on the smartphone through the touchscreen 60 or may enjoy music or video.

Here, a microphone 43 for voice input and a speaker 44 for voice output may be further provided.

The microphone 43 and the speaker 44 may be provided adjacent to the touchscreen 60 so as to enable voice call or video call through the touchscreen 60.

Further, an output unit 46 may be further provided to enable connection of, e.g., an earphone, so as to avoid annoying others while listening to music.

The output unit 46 may externally output images as well as sound outputted from the external device 200 by the touchscreen 60.

The touchscreen 60 is not limited as having the purpose of controlling the external device 200 and may also be used for various entry purposes for choosing or driving design information desired to be driven or entry of text to be displayed on the flexible display panel 10.

Or, according to another embodiment of the present invention, the smart display wear 1 may have a motion sensor 23 so that the smart display wear 1 may be connected with a game player to be played. To that end, multiple motion sensors 23 may be provided at multiple positions of the garment 100 to detect the wearer's motion.

The controller 30 may be provided to deliver information on the motion of the wearer detected by the motion sensor 23 to the external device 200 connected via the connector 41.

Meanwhile, according to another embodiment of the present invention, the smart display wear 1 may include a location sensor 25 for the wearer's safety.

In other words, the location sensor 25 may be provided on a side of the garment 100 to detect the location of the garment 100.

A transceiver 42 is provided to transmit or receive the result detected by the location sensor 25 with an outside. Here, the outside may be, e.g., a server of a mobile communication service provider. For example, in a case where the location sensor 25 is attached onto smart display wear 1 for kid requiring protection, the location sensor 25 may detect the wearer's location at every set period or when an event occurs and send the location information and smartphone number information to the mobile communication provider's server so that the location may be delivered to a smartphone registered by a guardian.

Further, according to another embodiment of the present invention, the smart display wear 1 may further include a caller 45 enabling emergency call when the wearer is at emergency.

The caller 45 is provided for the wearer to be able to select the emergency call by one or more methods of voice or button manipulation. For example, in a case where a voice speaking out ‘112’ or ‘119’ is entered to the microphone 43 or an emergency button provided at a predetermined position is pressed, the caller 45 enables the emergency to be delivered or known to a corresponding organization or portable terminal of a guardian whose contact information has been previously entered.

Thus, when the caller 45 is selected, the controller 30 sends the result of detection of the location sensor 25, along with a call signal, to the outside via the transceiver 42, allowing for effective response.

According to another embodiment of the present invention, the smart display wear 1 has functionality for preventing the wearer's wallet, mobile phone, or belongings from being lost or stolen.

To that end, the smart display wear 1 may further include a first signaling unit provided on a side of the garment 100 and a second signaling unit detachably provided in a belonging, such as a wallet, put in the garment and connected with the first signaling unit only within a predetermined distance.

The distance between the first signaling unit and the second signaling unit may be adjusted as necessary, and the distance may lie, e.g. 3 m to 5 m.

The second signaling unit may be provided in a sticker type attached or detached from the belonging, or the second signaling unit may be provided in a card type, accessory type (mobile phone ring or key ring, etc.) or any other various types.

When the first signaling unit loses connection with the second signaling unit, the controller 30 determines that the belonging is lost and displays the same on the flexible display panel 10. Or, the controller 30 may issue a voice alert through the speaker 44.

Meanwhile, the present invention includes a power supply 50 supplying power to the flexible display panel 10.

Various methods may be adopted in preparation for when the power supply 50 is discharged. For example, the power supply 50 may be detachably exchanged, the power supply 50 may be charged by an external power source via a wired connection, e.g., cable, or the power supply 50 ay be charged by a solar cell or panel provided on the outside of the garment 100 or by a tiny wind turbine.

While the inventive concept has been shown and described with reference to exemplary embodiments thereof, it will be apparent to those of ordinary skill in the art that various changes in form and detail may be made thereto without departing from the spirit and scope of the inventive concept as defined by the following claims.

What is claimed is:

1. Smart display wear, comprising:
a garment having a flexible display panel attached or fastened onto a whole or part of an outside of a lining of the garment or configuring the garment itself; and
a controller storing, as an image or video, design information including various colors or textures for each portion of the garment and outputting the design information to the flexible display panel according to a setting.

2. The smart display wear of claim 1, wherein the flexible display panel is divided into a plurality of panel areas independently separated from each other, and wherein the controller independently controls each of the panel areas.

3. The smart display wear of claim 1, further comprising a temperature sensor provided at, at least, one or more positions on the lining or the flexible display panel to measure a body temperature of a wearer of the garment, wherein the controller displays a temperature measurement sensed by the temperature sensor at a predetermined position on the flexible display panel.

4. The smart display wear of claim 3, wherein when the temperature measurement is not less than a set temperature previously entered and stored, the controller displays a color, flickering, or other alerts on the flexible display panel to externally notify.

5. The smart display wear of claim 1, further comprising:
a connector connective with an external device by one or more wired or wireless methods;
a touchscreen provided on a side of the garment to conduct input and output on the external device;
a microphone for inputting a voice; and
a speaker for outputting a voice.

6. The smart display wear of claim 5, further comprising an output unit for externally outputting an image and voice outputted from the external device by the touchscreen.

7. The smart display wear of claim 1, further comprising a connector connective with an external device by one or more wired or wireless methods; and motion sensors provided at multiple positions of the garment to detect a wearer's motion, wherein the wearer's motion detected by the motion sensors is delivered to an external device via the connector.

8. The smart display wear of claim 1, further comprising:
a location sensor provided on a side of the garment to detect a location of the garment,
a transceiver externally transmitting a result of detection by the location sensor so that a location of the wearer may be identified.

9. The smart display wear of claim 8, further comprising a caller allowing a wearer to select an emergency call by a method including one or more of a voice or button manipulation, wherein when the caller is selected, the controller externally transmits the result of detection by the location sensor, along with a call signal, via the transceiver.

10. The smart display wear of claim 1, further comprising a power supply supplying power to the flexible display panel, wherein the power supply may be used by any one of methods for detachably exchanging, charging via a wired connection with an external power source, charging by a solar cell, or charging by a tiny wind turbine.

11. The smart display wear of claim 1, further comprising a connector connectible with an external device by one or more wired or wireless methods to enable transmission or reception of the design information with the connected external device.

12. The smart display wear of claim 1, further comprising:
a first signaling unit provided on a side of the garment; and
a second signaling unit detachably provided on a belonging, such as a wallet, put in the garment to be connectible with the first signaling unit only within a predetermined distance, wherein when the first signaling unit loses connection with the second signaling unit, the controller determines that the belonging is lost and displays the same on the flexible display panel.
